SEQUENCE LISTING (13 sequences)

SEQ ID NO: 1 - DNA, 3870 bp, Aspergillus niger
  exon (1)..(201)
  Intron (202)..(270)
  exon (271)..(637)
  Intron (638)..(694)
  exon (695)..(838)
  Intron (839)..(889)
  exon (890)..(3138)
  Intron (3139)..(3186)
  exon (3187)..(3870)
atgacccgccccgaggacgacctggcctatggccagtactaccaggactccgcccggggagcctcttccggagactcctccaggggtctgagcgacacttttaagaagctgaagcagacttacaagtcccaccagtcgcaacagggctcttcccagcaatctcagcagtcgcagcaatcccagtccgccagctactacaatgtaagttgttgcctgtacctggtcacctccccgatccccggtactaaccgacccgttccaaactcccagacttcgaaccagacctaccagtcccaaggtccctcgcagtcccagcaataccatcctcagcagcaacaacaacaacagcagcaacaaccccatccgtcgaaaccgcagaagcaggacaaattttccggcttgtttggcaagctggaagaactcggcaatgaggtggcacagaaactgggtaccgcgctcgacccccaggcgtatgccgagtatggcgctccaaagccgcagaccgagaaccgcttcgggagctttgcggccccgcgtcagggtaacgaggtcaagtggcacgtggatggttgcgcctacttttatgctgtgtccaaggcattggagagtgccaaggattatatttggattctggactgtaggtacccaggggactgctgtgttggaggggcatgagactgactatgcattttagggtggctctctccggaactttacctgagacgaccccccgcaaagcacgaacagtaccggctggatcggatgctgttggctgcggcgcagcgcggagtccgggtgaacatcattgtgtacaaggaggtgacgcaggcactgacccgtatgttttgtgcgtctgttgcgtgaaccgtcaaactgaccctactggcagtctcctcacaccacaccaagcaccatctggaagacctccatgaaaacattgcagtattccgtcaccccgatcacctgcccgaccgtcaggaactcgaggcgtccatccatacgtctctccagaacttgtccctcgatgccggcaaccttgccaagatgtccgaagacgccatcaagggcatctacggcatgcacgaggatgtgattctgtactgggctcaccacgagaagctttgcctcattgatggccgcattgcgttcatgggtggtctggatatgtgctttggccgctgggacaccaaccagcatgaactggccgatgttcacggtcaggacctgaacaagattgtcttccccggtcaggactacaacaacgcccgagtgagtgatttccacgacgttgcccactgggagcagaaccagctggaccgcaaggacacttctcgcatgggctggtccgatatttcggtcagtttgcacggcccggtcgtcgaggatctgaggaagcactttgttcagcggtggaacttcatctatgactccaagtaccagtcgcgcaacaactcgagatacgccagattggccctgtacggccggccgacctcaggcccccagcagcagcaagggccccaacagggtggtcaggcccagaaaccgcccgcgtcgcctcagcctggtgccactgggcctcccccaccgagctggcaacagcaggcagcgtctccccagcctggggcaaatcctggtcctcctgctcctagctggcagcaacaggcagctccgtcgcagcctagcgcccaggcacctagttccagcagctcttctaccccaagctggcagcagcagcagaccggagttgccagcaacactcagccttccagcactgccaaccccgcgacacctacctggcagcagcaggcaccgacacctcaacagggaggctacgcagccagtccttcccccaacccgagcagccaggagaagcccagctggcaacagcagcctgcgcagcccagcggttaccaaccccaggcacaaaccactggcagccaggagaagcccagctggcaacagcagagctctgagcctcctgcgtactcggcccacccacagcagcactacacttacagtggtgactcgttccccccaccccctcctggtcctccgccagcccagaactctgtgcaggcgtcttaccaggcgtacaacccccagcagccgtcgcctcagaaccagacacccacccaaggccagagtcagactccttactatccgcctcccccgaaccaggaagtccaccactcgcaaacacgcggtattcacgacgcgcaccagagcggatatggcgactctgagaggggcttcaacccccgccgtctgcgtgagaacttcatggactacggcaacgtcctgcgtggcgagttggcaggccagatccatcagtaccaggatcggttctccactcatggccgtcaggttaaccagccccgtggtaacatgacctgccagatcgtgcgcagctgctcgaagtggagtaacggcactccgaccgagcactccattcaggatgcgtatgctgcggtcattcgcaacagtcagcactttatctacattgagaaccagttcttcatcacagcgaccggtgacgcgcagaagccggtggagaacaagatcggtgttgcgattgtggagcgcattctgcgcgctgcccgtgctggtgagaagttcaagatcatcgtcgtgattccctccgtcccctgctttgccggagatttgagcgatgaatccacccttggtacccgcgccatcatggaattccagtacaactgcatcaaccgcggaggcagcagcatcatggagatgattgccaaggagggattcaacccgatggactacatccggttctataacctgcgtaactacgaccgcatcaatgtcagcggcccgctgatgcaggctgagcagagcagcggcgtcaattacgaggatgcccgcaaacagcacgatgtgactaccggcggccctggtggttatggtcctggtgctccgcgggcagctttcgacaccaccgcgccttaccagcagtaccagcaagctgcccagcaggtgggcggcaagtctggccagtgggatagtgtgagcagctgctacatgctcaatggccctgatattcgcaatgtgccctggaacggacctccggaggccgagattgatgcgtttgtcaccgaggaactctatgttcactccaaggtacgttagccattcacatggtataagtgcagaactaacaagatgcaggtgatgattgctgacgaccgtgttgccattgtcggatcggctaacttgaacgaccgctctcaactgggaactcacgactcggaaattgccatcgtcattgaggactacacccctgtgcagtcccgcatgaacggccagccttggactgccagccggttcgctacctccctccgtcgtcagctgttccgcaagcacctgggactgctgccaccacaggacatggagcggccggacggcaacttcgagccagtgggcgttcccaacaccaccgacttcgagtcacccgagagccagattgtggccgatccgctggcggatacgctgcacagtatgtggaacacgcgggctcggacgaacacggaggtgttccgcaaggtcttccactcggttccggacgactcggtgcgcaactgggctacgtacaaggagttctacggatactacttccacaacgcggacaagcaggcgtatggcgaggacgagtccagacctgctcgctacaagtatgggcacgtggtccgcgacgacttccctccgggcccggagggtgtcaggcaagtcaaagaactgctcagccaggtcaagggcacgttggtggagatgcctttgatgttcctgattgaggaggatgtggcgaaggaggggttgacgctgaatgagattacggagccaatctacacttga

SEQ ID NO: 2 - DNA, 3645 bp, Aspergillus niger
  CDS (1)..(3642): Phospholipase-encoding cDNA
atgacccgccccgaggacgacctggcctatggccagtactaccaggactccgcccggggagcctcttccggagactcctccaggggtctgagcgacacttttaagaagctgaagcagacttacaagtcccaccagtcgcaacagggctcttcccagcaatctcagcagtcgcagcaatcccagtccgccagctactacaatacttcgaaccagacctaccagtcccaaggtccctcgcagtcccagcaataccatcctcagcagcaacaacaacaacagcagcaacaaccccatccgtcgaaaccgcagaagcaggacaaattttccggcttgtttggcaagctggaagaactcggcaatgaggtggcacagaaactgggtaccgcgctcgacccccaggcgtatgccgagtatggcgctccaaagccgcagaccgagaaccgcttcgggagctttgcggccccgcgtcagggtaacgaggtcaagtggcacgtggatggttgcgcctacttttatgctgtgtccaaggcattggagagtgccaaggattatatttggattctggactggtggctctctccggaactttacctgagacgaccccccgcaaagcacgaacagtaccggctggatcggatgctgttggctgcggcgcagcgcggagtccgggtgaacatcattgtgtacaaggaggtgacgcaggcactgaccctctcctcacaccacaccaagcaccatctggaagacctccatgaaaacattgcagtattccgtcaccccgatcacctgcccgaccgtcaggaactcgaggcgtccatccatacgtctctccagaacttgtccctcgatgccggcaaccttgccaagatgtccgaagacgccatcaagggcatctacggcatgcacgaggatgtgattctgtactgggctcaccacgagaagctttgcctcattgatggccgcattgcgttcatgggtggtctggatatgtgctttggccgctgggacaccaaccagcatgaactggccgatgttcacggtcaggacctgaacaagattgtcttccccggtcaggactacaacaacgcccgagtgagtgatttccacgacgttgcccactgggagcagaaccagctggaccgcaaggacacttctcgcatgggctggtccgatatttcggtcagtttgcacggcccggtcgtcgaggatctgaggaagcactttgttcagcggtggaacttcatctatgactccaagtaccagtcgcgcaacaactcgagatacgccagattggccctgtacggccggccgacctcaggcccccagcagcagcaagggccccaacagggtggtcaggcccagaaaccgcccgcgtcgcctcagcctggtgccactgggcctcccccaccgagctggcaacagcaggcagcgtctccccagcctggggcaaatcctggtcctcctgctcctagctggcagcaacaggcagctccgtcgcagcctagcgcccaggcacctagttccagcagctcttctaccccaagctggcagcagcagcagaccggagttgccagcaacactcagccttccagcactgccaaccccgcgacacctacctggcagcagcaggcaccgacacctcaacagggaggctacgcagccagtccttcccccaacccgagcagccaggagaagcccagctggcaacagcagcctgcgcagcccagcggttaccaaccccaggcacaaaccactggcagccaggagaagcccagctggcaacagcagagctctgagcctcctgcgtactcggcccacccacagcagcactacacttacagtggtgactcgttccccccaccccctcctggtcctccgccagcccagaactctgtgcaggcgtcttaccaggcgtacaacccccagcagccgtcgcctcagaaccagacacccacccaaggccagagtcagactccttactatccgcctcccccgaaccaggaagtccaccactcgcaaacacgcggtattcacgacgcgcaccagagcggatatggcgactctgagaggggcttcaacccccgccgtctgcgtgagaacttcatggactacggcaacgtcctgcgtggcgagttggcaggccagatccatcagtaccaggatcggttctccactcatggccgtcaggttaaccagccccgtggtaacatgacctgccagatcgtgcgcagctgctcgaagtggagtaacggcactccgaccgagcactccattcaggatgcgtatgctgcggtcattcgcaacagtcagcactttatctacattgagaaccagttcttcatcacagcgaccggtgacgcgcagaagccggtggagaacaagatcggtgttgcgattgtggagcgcattctgcgcgctgcccgtgctggtgagaagttcaagatcatcgtcgtgattccctccgtcccctgctttgccggagatttgagcgatgaatccacccttggtacccgcgccatcatggaattccagtacaactgcatcaaccgcggaggcagcagcatcatggagatgattgccaaggagggattcaacccgatggactacatccggttctataacctgcgtaactacgaccgcatcaatgtcagcggcccgctgatgcaggctgagcagagcagcggcgtcaattacgaggatgcccgcaaacagcacgatgtgactaccggcggccctggtggttatggtcctggtgctccgcgggcagctttcgacaccaccgcgccttaccagcagtaccagcaagctgcccagcaggtgggcggcaagtctggccagtgggatagtgtgagcagctgctacatgctcaatggccctgatattcgcaatgtgccctggaacggacctccggaggccgagattgatgcgtttgtcaccgaggaactctatgttcactccaaggtgatgattgctgacgaccgtgttgccattgtcggatcggctaacttgaacgaccgctctcaactgggaactcacgactcggaaattgccatcgtcattgaggactacacccctgtgcagtcccgcatgaacggccagccttggactgccagccggttcgctacctccctccgtcgtcagctgttccgcaagcacctgggactgctgccaccacaggacatggagcggccggacggcaacttcgagccagtgggcgttcccaacaccaccgacttcgagtcacccgagagccagattgtggccgatccgctggcggatacgctgcacagtatgtggaacacgcgggctcggacgaacacggaggtgttccgcaaggtcttccactcggttccggacgactcggtgcgcaactgggctacgtacaaggagttctacggatactacttccacaacgcggacaagcaggcgtatggcgaggacgagtccagacctgctcgctacaagtatgggcacgtggtccgcgacgacttccctccgggcccggagggtgtcaggcaagtcaaagaactgctcagccaggtcaagggcacgttggtggagatgcctttgatgttcctgattgaggaggatgtggcgaaggaggggttgacgctgaatgagattacggagccaatctacacttga

SEQ ID NO: 3 - protein, 1214 aa, Aspergillus niger
MTRPEDDLAYGQYYQDSARGASSGDSSRGLSDTFKKLKQTYKSHQSQQGSSQQSQQSQQSQSASYYNTSNQTYQSQGPSQSQQYHPQQQQQQQQQQPHPSKPQKQDKFSGLFGKLEELGNEVAQKLGTALDPQAYAEYGAPKPQTENRFGSFAAPRQGNEVKWHVDGCAYFYAVSKALESAKDYIWILDWWLSPELYLRRPPAKHEQYRLDRMLLAAAQRGVRVNIIVYKEVTQALTLSSHHTKHHLEDLHENIAVFRHPDHLPDRQELEASIHTSLQNLSLDAGNLAKMSEDAIKGIYGMHEDVILYWAHHEKLCLIDGRIAFMGGLDMCFGRWDTNQHELADVHGQDLNKIVFPGQDYNNARVSDFHDVAHWEQNQLDRKDTSRMGWSDISVSLHGPVVEDLRKHFVQRWNFIYDSKYQSRNNSRYARLALYGRPTSGPQQQQGPQQGGQAQKPPASPQPGATGPPPPSWQQQAASPQPGANPGPPAPSWQQQAAPSQPSAQAPSSSSSSTPSWQQQQTGVASNTQPSSTANPATPTWQQQAPTPQQGGYAASPSPNPSSQEKPSWQQQPAQPSGYQPQAQTTGSQEKPSWQQQSSEPPAYSAHPQQHYTYSGDSFPPPPPGPPPAQNSVQASYQAYNPQQPSPQNQTPTQGQSQTPYYPPPPNQEVHHSQTRGIHDAHQSGYGDSERGFNPRRLRENFMDYGNVLRGELAGQIHQYQDRFSTHGRQVNQPRGNMTCQIVRSCSKWSNGTPTEHSIQDAYAAVIRNSQHFIYIENQFFITATGDAQKPVENKIGVAIVERILRAARAGEKFKIIVVIPSVPCFAGDLSDESTLGTRAIMEFQYNCINRGGSSIMEMIAKEGFNPMDYIRFYNLRNYDRINVSGPLMQAEQSSGVNYEDARKQHDVTTGGPGGYGPGAPRAAFDTTAPYQQYQQAAQQVGGKSGQWDSVSSCYMLNGPDIRNVPWNGPPEAEIDAFVTEELYVHSKVMIADDRVAIVGSANLNDRSQLGTHDSEIAIVIEDYTPVQSRMNGQPWTASRFATSLRRQLFRKHLGLLPPQDMERPDGNFEPVGVPNTTDFESPESQIVADPLADTLHSMWNTRARTNTEVFRKVFHSVPDDSVRNWATYKEFYGYYFHNADKQAYGEDESRPARYKYGHVVRDDFPPGPEGVRQVKELLSQVKGTLVEMPLMFLIEEDVAKEGLTLNEITEPIYT

SEQ ID NO: 4 - DNA, 30 bp, Artificial sequence
  Primer spo14-1 (sense)
cggtggcggccgcattcaacaaccgagtga

SEQ ID NO: 5 - DNA, 31 bp, Artificial sequence
  Primer spo14-2 (antisense)
cgctccgactagttagatactagactagata

SEQ ID NO: 6 - DNA, 30 bp, Artificial sequence
  Primer spo14-3 (sense)
gtttaaaccactggcagccaggagaagccc

SEQ ID NO: 7 - DNA, 30 bp, Artificial sequence
  Primer spo14-4 (antisense)
ttaattaaaatgaaggaagagatggaagga

SEQ ID NO: 8 - DNA, 20 bp, Artificial sequence
  Forward primer (spo14-5)
cactggcagccaggagaagc

SEQ ID NO: 9 - DNA, 20 bp, Artificial sequence
  Reverse primer (spo14-6)
ggagtgctcggtcggagtgc

SEQ ID NO: 10 - DNA, 40 bp, Artificial sequence
  Primerpldex-1 (sense)
ggatttagtcttgatcggatccaccatgcgttggcctggt

SEQ ID NO: 11 - DNA, 40 bp, Artificial sequence
  Primerpldex-2 (antisense)
gaaatggattgattgtcacgtgttattcaatgctccagtc

SEQ ID NO: 12 - DNA, 20 bp, Artificial sequence
  Forward primer (pldexC-3)
atgcgttggcctggtaattt

SEQ ID NO: 13 - DNA, 20 bp, Artificial sequence
  Reverse primer (pldexC-4)
gatggtctcgtagacaaacg

(19) United States
(12) Patent Application Publication
(10) Pub. No.: US 2022/0251581 A1
(43) Pub. Date: Aug. 11, 2022

(54) MODIFIED FILAMENTOUS FUNGAL HOST CELL

(71) Applicant: Novozymes A/S, Bagsvaerd (DK)

(72) Inventor: Hiroaki Udagawa, Ichikawa (JP)

(73) Assignee: Novozymes A/S, Bagsvaerd (DK)

(21) Appl. No.: 17/630,476

(22) PCT Filed: Jul. 24, 2020
(86) PCT No.: PCT/EP2020/071004

§ 371 Date: Jan. 26, 2022

(30) Foreign Application Priority Data
Jul. 26, 2019 (EP) 19188683.7

(51) Int. Cl. C12N 15/80; C12N 1/14; C12P 21/02; C12N 9/16

(52) U.S. Cl. CPC C12N 15/80; C12N 1/145; C12R 2001/685; C12N 9/16; C12Y 301/04004; C12P 21/02

(57) ABSTRACT

The present invention relates to phospholipase D-inactivated filamentous fungal cells secreting a polypeptide of interest and methods of producing a secreted polypeptide of interest in said cells as well as methods of producing said cells.

### REFERENCE TO SEQUENCE LISTING

This application contains a Sequence Listing in computer readable form. The computer readable form is incorporated herein by reference.

### FIELD OF THE INVENTION

The present invention relates to modified phospholipase D-inactivated filamentous fungal cells secreting a polypeptide of interest, and methods of producing a secreted polypeptide of interest in said cells as well, as methods of producing said cells.

### BACKGROUND OF THE INVENTION

Recombinant gene expression in filamentous fungal host cells is a common method for production of polypeptides of interest, such as, enzymes and other valuable proteins. For industrial and commercial purposes, the productivity or product yield of filamentous fungal host strains is an important factor of production costs.

Ways of increasing the productivity or yield of a heterologous protein in filamentous fungal cells are always of commercial interest.

### SUMMARY OF THE INVENTION

The present invention is directed to genetically modified filamentous fungal host cells in which production of a native phospholipase has been inactivated. Inactivation of the phospholipase may be done by any suitable gene inactivation method known in the art. An example of a convenient way to eliminate or reduce phospholipase production is based on techniques of gene replacement or gene interruption of the phospholipase-encoding gene.

The inactivation of the phospholipase-encoding spo14 gene in an Aspergillus filamentous fungal host cell resulted in increased yield of a several heterologous secreted polypeptides of interest expressed in the cell, a glucoamylase (AGU) and a dextranase.

Accordingly, in a first aspect, the invention relates to filamentous fungal host cells comprising a heterologous polynucleotide encoding a secreted polypeptide of interest and comprising an inactivated spo14 gene or homologue thereof, wherein said spo14 gene or homologue thereof encodes a phospholipase D having an amino acid sequence at least 70% identical to SEQ ID NO:3; preferably at least 75%, 80%, 85%, 90%, 95%, 96%, 97%, 98% or most preferably at least 99% identical to SEQ ID NO:3.

The invention further provides methods for producing a heterologous secreted polypeptide of interest by cultivating a filamentous fungal host cell of the invention under conditions conducive for expression of the polypeptide of interest and, optionally, recovering the polypeptide of interest.

Accordingly, in a second aspect, the invention relates to methods of producing a secreted polypeptide of interest, said method comprising the steps of:
a) cultivating a filamentous fungal host cell comprising a heterologous polynucleotide encoding the secreted polypeptide of interest and comprising an inactivated spo14 gene or homologue thereof under conditions conducive to the expression of the secreted polypeptide of interest, wherein said spo14 gene or homologue thereof encodes a phospholipase D having an amino acid sequence at least 70% identical to SEQ ID NO:3, preferably at least 75%, 80%, 85%, 90%, 95%, 96%, 97%, 98% or most preferably at least 99% identical to SEQ ID NO:3; and, optionally,
b) recovering the secreted polypeptide of interest.

In a final aspect, the invention relates to methods of producing a filamentous fungal host cell having an improved yield of a secreted heterologous polypeptide of interest, said method comprising the following steps in no particular order:
a) transforming a filamentous fungal host cell with a heterologous polynucleotide encoding the secreted polypeptide of interest; and
b) inactivating an spo14 gene or a homologue thereof in the filamentous fungal host cell, wherein said spo14 gene or a homologue thereof encodes an phospholipase D having an amino acid sequence at least 70% identical to SEQ ID NO:3, preferably at least 75%, 80%, 85%, 90%, 95%, 96%, 97%, 98% or most preferably at least 99% identical to SEQ ID NO:3.

### Definitions

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

### DETAILED DESCRIPTION OF THE INVENTION

### Host Cells

The present invention relates to recombinant host cells comprising a polynucleotide of the present invention operably linked to one or more control sequences that direct the production and secretion of a heterologous polypeptide of interest.

A construct or vector comprising a polynucleotide is introduced into a host cell so that the construct or vector is maintained as a chromosomal integrant or as a self-replicating extrachromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell of the invention is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

In one aspect, the invention relates to methods of producing a filamentous fungal host cell having an improved yield of a secreted heterologous polypeptide of interest, said method comprising the following steps in no particular order:
a) transforming a filamentous fungal host cell with a heterologous polynucleotide encoding the secreted polypeptide of interest; and
b) inactivating an spo14 gene or a homologue thereof in the filamentous fungal host cell, wherein said spo14 gene or a homologue thereof encodes an phospholipase D having an amino acid sequence at least 70% identical to SEQ ID NO:3, preferably at least 75%, 80%, 85%, 90%, 95%, 96%, 97%, 98% or most preferably at least 99% identical to SEQ ID NO:3.

In another aspect, the invention relates to the resulting filamentous fungal host cells comprising a heterologous polynucleotide encoding a secreted polypeptide of interest and comprising an inactivated spo14 gene or homologue thereof, wherein said spo14 gene or homologue thereof encodes a phospholipase D having an amino acid sequence at least 70% identical to SEQ ID NO:3; preferably at least 75%, 80%, 85%, 90%, 95%, 96%, 97%, 98% or most preferably at least 99% identical to SEQ ID NO:3.

In a preferred embodiment of the aspects of the invention, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma; even more preferably the filamentous fungal host cell is an Aspergillus cell; preferably an Aspergillus awamori, Aspergillus foetidus, Aspergillus fumigatus, Aspergillus japonicus, Aspergillus nidulans, Aspergillus niger or an Aspergillus oryzae cell.

Preferably, the secreted polypeptide of interest is an enzyme; preferably the enzyme is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, xylanase, or beta-xylosidase; most preferably the secreted polypeptide of interest is a glucoamylase.

In a preferred embodiment of the invention, the phospholipase D comprises or consists of an amino acid sequence at least 70% identical to SEQ ID NO:3; preferably at least 75%, 80%, 85%, 90%, 95%, 96%, 97%, 98% or most preferably at least 99% identical to SEQ ID NO:3.

Preferably, the spo14 gene or homologue thereof comprises or consists of a genomic nucleotide sequence at least 70% identical to SEQ ID NO:1; preferably at least 75%, 80%, 85%, 90%, 95%, 96%, 97%, 98% or most preferably at least 99% identical to SEQ ID NO:1. Alternatively, the spo14 gene or homologue thereof comprises or consists of a genomic nucleotide sequence, the cDNA sequence of which is at least 70% identical to SEQ ID NO:2; preferably at least 75%, 80%, 85%, 90%, 95%, 96%, 97%, 98% or most preferably at least 99% identical to SEQ ID NO:2.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Removal or Reduction of Phospholipase D Activity

The present invention also relates to methods of producing a mutant of a parent cell, which comprises inactivating, disrupting or deleting a polynucleotide, or a portion thereof, encoding an phospholipase D polypeptide of the present invention, which results in the mutant cell producing less of the phospholipase D polypeptide than the parent cell when cultivated under the same conditions.

The mutant cell may be constructed by reducing or eliminating expression of the spo14 polynucleotide or a homologue thereof using methods well known in the art, for example, insertions, disruptions, replacements, or deletions. In a preferred aspect, the polynucleotide is inactivated. The polynucleotide to be modified or inactivated may be, for example, the coding region or a part thereof essential for activity, or a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the spo14 polynucleotide or homologue thereof may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

Methods for deleting or disrupting a targeted gene are described, for example, by Miller, et al (1985. Mol. Cell. Biol. 5:1714-1721); WO 90/00192; May, G. (1992. Applied Molecular Genetics of Filamentous Fungi. J. R. Kinghorn and G. Turner, eds., Blackie Academic and Professional, pp. 1-25); and Turner, G. (1994. Vectors for Genetic Manipulation. S. D. Martinelli and J. R. Kinghorn, eds., Elsevier, pp. 641-665).

An example of a convenient way to eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention also relates to methods of inhibiting the expression of a polypeptide having phospholipase D activity in a cell, comprising administering to the cell or expressing in the cell a double-stranded RNA (dsRNA) molecule, wherein the dsRNA comprises a subsequence of an spo14 polynucleotide or homologue thereof. In a preferred aspect, the dsRNA is about 15, 16, 17, 18, 19, 20, 21, 22, 23, 24, 25 or more duplex nucleotides in length.

The dsRNA is preferably a small interfering RNA (siRNA) or a micro RNA (miRNA). In a preferred aspect, the dsRNA is small interfering RNA for inhibiting transcription. In another preferred aspect, the dsRNA is micro RNA for inhibiting translation.

The present invention also relates to such double-stranded RNA (dsRNA) molecules, comprising a portion of the mature polypeptide coding sequence of SEQ ID NO: 1 for inhibiting expression of the polypeptide in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi); see, for example, U.S. Pat. No. 5,190,931.

The dsRNAs of the present invention can be used in gene-silencing. In one aspect, the invention provides methods to selectively degrade RNA using a dsRNAi of the present invention. The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art; see, for example, U.S. Pat. Nos. 6,489,127; 6,506,559; 6,511,824 and 6,515,109.

The phospholipase D polypeptide-deficient mutant cells are particularly useful as host cells for expression of heterologous secreted polypeptides.

The methods used for cultivation and purification of the product of interest may be performed by methods known in the art.

### Methods of Production

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

One aspect of the invention relates to methods of producing a secreted polypeptide of interest, said method comprising the steps of:
a) cultivating a filamentous fungal host cell comprising a heterologous polynucleotide encoding the secreted polypeptide of interest and comprising an inactivated spo14 gene or homologue thereof under conditions conducive to the expression of the secreted polypeptide of interest, wherein said spo14 gene or homologue thereof encodes a phospholipase D having an amino acid sequence at least 70% identical to SEQ ID NO:3, preferably at least 75%, 80%, 85%, 90%, 95%, 96%, 97%, 98% or most preferably at least 99% identical to SEQ ID NO:3; and, optionally,
b) recovering the secreted polypeptide of interest.

In a preferred embodiment, the filamentous fungal host cell is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma; even more preferably the filamentous fungal host cell is an Aspergillus cell; preferably an Aspergillus awamori, Aspergillus foetidus, Aspergillus fumigatus, Aspergillus japonicus, Aspergillus nidulans, Aspergillus niger or an Aspergillus oryzae cell.

Preferably, the secreted polypeptide of interest is an enzyme; preferably the enzyme is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase; most preferably the secreted polypeptide of interest is a glucoamylase.

In a preferred embodiment of the invention, the phospholipase D comprises or consists of an amino acid sequence at least 70% identical to SEQ ID NO:3; preferably at least 75%, 80%, 85%, 90%, 95%, 96%, 97%, 98% or most preferably at least 99% identical to SEQ ID NO:3.

Preferably, the spo14 gene or homologue thereof comprises or consists of a genomic nucleotide sequence at least 70% identical to SEQ ID NO:1; preferably at least 75%, 80%, 85%, 90%, 95%, 96%, 97%, 98% or most preferably at least 99% identical to SEQ ID NO:1. Alternatively, the spo14 gene or homologue thereof comprises or consists of a genomic nucleotide sequence, the cDNA sequence of which is at least 70% identical to SEQ ID NO:2; preferably at least 75%, 80%, 85%, 90%, 95%, 96%, 97%, 98% or most preferably at least 99% identical to SEQ ID NO:2.

### EXAMPLES

### Materials and Methods

Molecular cloning techniques are described in Sambrook, J., Fritsch, E. F., Maniatis, T. (1989) Molecular cloning: a laboratory manual (2nd edn.) Cold Spring Harbor Laboratory, Cold Spring Harbor, N.Y.

### Enzymes

Enzymes for DNA manipulations (e.g. restriction endonucleases, ligases etc.) are obtainable from New England Biolabs, Inc. and were used according to the manufacturer's instructions.

### Media and Solutions

AMG trace metals solution was composed of 0.3 g of citric acid, 0.68 g of ZnCl₂, 0.25 g of CuSO₄.5H₂O, 0.024 g of NiCl₂.6H₂O, 1.39 g of FeSO₄.7H₂O, 1.356 g of MnSO₄.5H₂O, and deionized water to 1 liter.

COVE-N-glyX plates were composed of 218 g of xylitol, 10 g of glycerol, 2.02 g of KNO₃, 50 ml of COVE salt solution, 25 g of Noble agar, and deionized water to 1 liter.

COVE medium was composed of 342.3 g of sucrose, 20 ml of 50×COVE salts solution, 10 ml of 1 M acetamide, 10 ml of 1.5 M CsCl₂, 25 g of Noble agar, and deionized water to 1 liter.

COVE2 medium was composed of 30 g of sucrose, 20 ml of 50×COVE salts solution, 10 ml of 1 M acetamide, 25 g of Noble agar, and deionized water to 1 liter.

COVE-N plates were composed of 342.3 g of sucrose, 20 ml of COVE salt solution, 3 g of NaNO₃, 30 g of Noble agar, and deionized water to 1 liter.

COVE-N top agarose was composed of 342.3 g of sucrose, 20 ml of COVE salt solution, 3 g of NaNO₃, 10 g of low melt agarose, and deionized water to 1 liter.

COVE-N JP plates were composed of 30 g of sucrose, 20 ml of COVE salt solution, 3 g of NaNO₃, 30 g of Noble agar, and deionized water to 1 liter.

COVE salt solution was composed of 26 g of KCl, 26 g of MgSO₄.7H₂O, 76 g of KH₂PO₄, 50 ml of COVE trace metals solution, and deionized water to 1 liter.

COVE trace metals was composed of 0.04 g of Na₂B₄O₇.10H₂O, 0.4 g of CuSO₄.5H₂O, 1.2 g of FeSO₄.7H₂O, 1.0 g of MnSO₄.5H₂O, 0.8 g of Na₂MoO₄.2H₂O, 10 g of ZnSO₄.7H₂O, and deionized water to 1 liter.

LB medium was composed of 10 g of tryptone, 5 g of yeast extract, 5 g of sodium chloride, and deionized water to 1 liter.

LB plus ampicillin plates were composed of 10 g of tryptone, 5 g of yeast extract, 5 g of sodium chloride, 15 g of Bacto agar, ampicillin at 100 μg per ml, and deionized water to 1 liter.

MSS medium was composed of 70 g of sucrose, 100 g of soy bean powder, three drops of pluronic antifoam, and deionized water to 1 liter; pH adjusted to 6.0.

MU-1 medium without urea was composed of 260 g of maltodextrin, 3 g of MgSO₄.7H₂O, 6 g of K₂SO₄, 5 g of KH₂PO₄, 0.5 ml of AMG trace metals solution, a few drops of antifoam, and deionized water to 1 liter; pH adjusted to 4.5.

MU-1 glu medium without urea was composed of 260 g of glucose, 3 g of MgSO₄.7H₂O, 6 g of K₂SO₄, 5 g of KH₂PO₄, 0.5 ml of AMG trace metals solution, a few drops of antifoam, and deionized water to 1 liter; pH adjusted to 4.5.

50% Urea was composed of 500 g of urea and deionized water to 1 liter.

YPG medium was composed of 10 g of yeast extract, 20 g of Bacto peptone, 20 g of glucose, and deionized water to 1 liter.

STC was composed of 0.8 M sorbitol, 25 mM or 50 mM Tris pH 8, and 25 mM or 50 mM CaCl₂.

SPTC was composed of 40% polyethyleneglycol 4000 (PEG4000) in STC buffer.

SOC medium was composed of 20 g of tryptone, 5 g of yeast extract, 0.5 g of NaCl, 10 ml of 250 mM KCl, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.

Purchased Material (E. coli, Plasmid and Kits)

E. coli DH5-alpha (Toyobo) was used for plasmid construction and PCR amplification. The commercial plasmids pBluescript II SK- (Stratagene #212206) were used for cloning of PCR fragments. Amplified plasmids were recovered with Qiagen Plasmid Kit (Qiagen). Ligation was done with DNA ligation kit (Takara) or T4 DNA ligase (Boehringer Mannheim). Polymerase Chain Reaction (PCR) was carried out with Expand™ PCR system (Boehringer Mannheim). QIAquick™ Gel Extraction Kit (Qiagen) was used for the purification of PCR fragments and extraction of DNA fragment from agarose gel.

### Strains

The expression host strains Aspergillus niger O73TYS and O73P66 were isolated by Novozymes and are derivatives of Aspergillus niger NN049184, which was isolated from soil. O73TYS and O73P66 were genetically modified to disrupt expression of amyloglucosidase activities and alpha-amylase activities followed by introducing Aspergillus niger cytosine deaminase gene (fcy1).

### Plasmids

The plasmid pHUda801 is described in example 4 in WO2012160093. The plasmid pRika147 for the vector of expression of the enzyme genes is described in example 9 in WO2012160093.

Transformation of Aspergillus niger

Transformation of the parent Aspergillus niger host cell was achieved using the general methods known for transformation in filamentous fungi, as described in the Yelton et al., “Transformation of Aspergillus nidulans by using a trpC plasmid,” Proc Natl Acad Sci USA. 1984 March; 81(5):1470-4, and as follows:

The Aspergillus niger host strain was inoculated to 100 ml of YPG medium supplemented with 10 mM uridine in case the host strain is a pyrG deficient mutant, and incubated for 16 hrs at 32° C. at 80 rpm. Pellets were collected and washed with 0.6 M KCl, and resuspended 20 ml 0.6 M KCl containing a commercial β-glucanase product (GLUCANEX™, Novozymes A/S, Bagsværd, Denmark) at a final concentration of 20 mg per ml. The suspension was incubated at 32° C. at 80 rpm until protoplasts were formed, and then washed twice with STC buffer. The protoplasts were counted with a hematometer and resuspended and adjusted in an 8:2:0.1 solution of STC:STPC:DMSO to a final concentration of 2.5×10⁷ protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. COVE-N top agarose, the mixture was poured onto the minimum medium and the plates were incubated at 30° C. for 5 days.

PCR amplification

| | |
|---|---|
| 5x PCR buffer (incl.MgCl2) | 20 μl |
| 2.5 mM dNTP mix | 10 μl |
| Forward primer (100 μM) | 1 μl |
| Reverse primer (100 μM) | 1 μl |
| Expand High Fidelity polymerase (Roche) | 1 μl |
| Template DNA (50-100 ng/μl) | 1 μl |
| Distilled water to | 100 μl |

PCR conditions

| | | |
|---|---|---|
| 94 C. | 2 min | 1 cycle |
| 92 C. | 1 min | |
| 55 C. | 1 min | 30 cycles |
| 72 C. | 1-2 min | |
| 72 C. | 7 min | 1 cycle |

### Shake Flask Cultivation for Reporter Enzyme Production

Spores of the selected transformants were inoculated in 100 ml of MSS media and cultivated at 30 C for 3 days. 10% of seed culture was transferred to MU-1 or MU-1 glu medium in shake flasks and cultivated at 32 C for 6 days. The supernatant was obtained by centrifugation. Culture supernatant after centrifugation was used for enzyme assay.

### Lab-Scale Tank Cultivation for Glucoamylase Production

Fermentation was done as fed-batch fermentation ((H. Pedersen 2000). Selected strains were pre-cultured in liquid media then grown mycelia were transferred to the tanks for further cultivation of enzyme production. Cultivation was done at pH 4.75 at 34 C for 8 days with the feeding of glucose and ammonium without over-dosing which prevents enzyme production. Culture whole broth was used for enzyme assay

### Southern Hybridization

Mycelia of the selected transformants were harvested from overnight culture in 3 ml YPG medium, rinsed with distilled water. Ground mycelia were subject to genome DNA preparation using FastDNA SPIN Kit for Soil (MP Biomedicals) follows by manufacture's instruction. Non-radioactive probes were synthesized using a PCR DIG probe synthesis kit (Roche Applied Science, Indianapolis Ind.) followed by manufacture's instruction. DIG labeled probes were gel purified using a QIAquick™ Gel Extraction Kit (QIAGEN Inc., Valencia, Calif.) according to the manufacturer's instructions.

Five micrograms of genome DNA was digested with appropriate restriction enzymes completely for 16 hours (40 μl total volume, 4 U enzyme/μl DNA) and run on a 0.8% agarose gel. The DNA was fragmented in the gel by treating with 0.2 M HCl, denatured (0.5M NaOH, 1.5M NaCl) and neutralized (1M Tris, pH7.5; 1.5M NaCl) for subsequent transfer in 20×SSC to Hybond N+ membrane (Amersham). The DNA was UV cross-linked to the membrane and prehybridized for 1 hour at 42° C. in 20 ml DIG Easy Hyb (Roche Diagnostics Corporation, Mannheim, Germany). The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker.

### Bradford Assay (Whole Protein Determination)

The Bradford assay, a colorimetric protein assay, is based on an absorbance shift of the dye Coomassie Brilliant Blue G-250 in which under acidic conditions the red form of the dye is converted into its bluer form to bind to the protein being assayed. The binding of the dye to the protein stabilizes the blue anionic form. The increase of absorbance at 595 nm is proportional to the amount of bound dye, and thus to the amount (concentration) of protein present in the sample. Enzyme samples were diluted by distilled water appropriately and were measured their protein amounts by use of Quick Start™ Bradford Protein Assay (Bio-Rad inc.) followed by manufactures' instruction.

### Glucoamylase Activity

Glucoamylase activity is measured in AmyloGlucosidase Units (AGU). The AGU is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes. An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

### Amyloglucosidase Incubation:

Substrate: maltose 23.2 mM
Buffer: acetate 0.1 M
pH: 4.30±0.05
Incubation temperature: 37° C.±1
Reaction time: 5 minutes
Enzyme working range: 0.5-4.0 AGU/mL

### Color Reaction:

### GlucDH: 430 U/L

### Mutarotase: 9 U/L

### NAD: 0.21 mM

Buffer: phosphate 0.12 M; 0.15 M NaCl
pH: 7.60±0.05
Incubation temperature: 37° C.±1
Reaction time: 5 minutes

### Wavelength: 340 nm

### Example 1: Construction of Plasmid pHUda2368, a Vector for Targeted Gene Disruption of Aspergillus niger Spo14 Gene

Plasmid pHUda2368 was constructed to contain 5′ and 3′ flanking regions for the Aspergillus niger phospholipase D (spo14) gene separated by the A. nidulans orotidine-5′-phosphate decarboxylase gene (pyrG) as a selectable marker with its terminator repeats, and the human Herpes simplex virus 1 (HSV-1) thymidine kinase gene. The HSV-1 thymidine kinase gene lies 3′ of the 3′ flanking region of the spo14 gene, allowing for counter-selection of Aspergillus niger transformants that do not correctly target to the spo14 gene locus. The plasmid was constructed in several steps as described below.

A PCR product containing the 5′ flanking region of A. niger spo14 was generated using the following primers:

Primer spo14-1
(sense):
(SEQ ID NO: 4)
5′-CGGTGGCGGCCGCATTCAACAACCGAGTGA-3′
Primer spo14-2
(antisense):
(SEQ ID NO: 5)
5′-CGCTCCGACTAGTTAGATACTAGACTAGATA-3′

The desired fragment was amplified by PCR in a reaction composed of approximately 100 ng of genome DNA of Aspergillus niger O73TYS, 1 μl of Expand High Fidelity polymerase (Roche), 100 μM of primer spo14-1, 100 μM of primer spo14-2, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 2 minutes; 1 cycle at 72° C. for 7 minutes; and a 4° C. hold. The resulting 2,554 bp PCR fragment was purified by 0.8% agarose gel electrophoresis using TAE buffer, excised from the gel, and extracted using a QIAQUICK® Gel Extraction Kit. The purified 2,554 bp PCR fragment was digested by NotI and SpeI.

Plasmid pHUda801 (Example 4 in WO 2012160093 A1) was digested with Not I and SpeI, and purified by 0.8% agarose gel electrophoresis using TAE buffer, where a 9,558 bp fragment was excised from the gel and extracted using a QIAQUICK® Gel Extraction Kit. The 9,558 bp fragment was ligated to the 2,554 bp PCR fragment in a reaction composed of 1 μl of the 9,558 bp fragment, 3 μl of the 2,554 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5a chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pH Uda801-5′spo14.

A PCR product containing the 3′ flanking region of A. niger spo14 was generated using the following primers:

Primer spo14-3
(sense):
(SEQ ID NO: 6)
5′-GTTTAAACCACTGGCAGCCAGGAGAAGCCC-3′
Primer spo14-4
(antisense):
(SEQ ID NO: 7)
5′-TTAATTAAAATGAAGGAAGAGATGGAAGGA-3′

The desired fragment was amplified by PCR in a reaction composed of approximately 100 ng of genome DNA of Aspergillus niger O73TYS, 1 μl of Expand High Fidelity polymerase (Roche), 100 μM of primer spo14-3, 100 μM of primer spo14-4, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 2 minutes; 1 cycle at 72° C. for 7 minutes; and a 4° C. hold. The resulting 2,980 bp PCR fragment was purified by 0.8% agarose gel electrophoresis using TAE buffer, excised from the gel, and extracted using a QIAQUICK® Gel Extraction Kit. The purified 2,980 bp PCR fragment was digested by PmeI and PacI.

Plasmid pHUda801-5′spo14 was digested with PmeI and PacI, and purified by 0.8% agarose gel electrophoresis using TAE buffer, where a 10,091 bp fragment was excised from the gel and extracted using a QIAQUICK® Gel Extraction Kit. The 10,091 bp fragment was ligated to the 2,980 bp PCR fragment in a reaction composed of 1 μl of the 10,091 bp fragment, 3 μl of the 2,980 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5a chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda2368.

### Example 2: The Aspergillus niger Spo14 Gene Disruption in O73P66

Protoplasts of Aspergillus niger strain O73P66 were prepared by cultivating the strain in 100 ml of YPG medium supplemented with 10 mM uridine at 32° C. for 16 hours with gentle agitation at 80 rpm. Pellets were collected and washed with 0.6 M KCl, and resuspended 20 ml 0.6 M KCl containing a commercial f3-glucanase product (GLUCANEX™, Novozymes A/S, Bagsværd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 10 μg of pHUda2368 was added to 0.3 ml of the protoplast suspension, mixed gently, and incubated on ice for 30 minutes. Three ml of SPTC was added and the protoplast suspension was incubated for 20 minutes at 37° C. After the addition of 12 ml of 50° C. COVE-N top agarose, the mixture was poured onto the COVE-N plates and the plates were incubated at 30° C. for 7 days. The grown transformants were transferred with sterile toothpicks to Cove-N JP plates supplemented with 1.5 μM 5-Flouro-2-deoxyuridine (FdU), an agent which kills cells expressing the Herpes simplex virus (HSV) thymidine kinase gene (TK) harboring in pHUda2368. Single spore isolates were transferred to COVE-N-glyX plates.

Possible transformants of Aspergillus niger strain O73P66 containing the pHUda2368 to disrupt spo14 gene were screened by Southern analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) follows by manufacture's instruction.

Southern blot analysis was performed to confirm the disruption of the spo14 gene locus. Five μg of genomic DNA from each transformant were digested with SpeI and SphI. The genomic DNA digestion reactions were composed of 5 μg of genomic DNA, 1 μl of SpeI, 1 μl of SphI, 2 μl of 10×NEBuffer 4, and water to 20 μl. Genomic DNA digestions were incubated at 37° C. for approximately 16 hours. The digestions were submitted to 0.8% agarose gel electrophoresis using TAE buffer and blotted onto a hybond N+ (GE Healthcare Life Sciences, Manchester, N.H., USA) using a TURBOBLOTTER® for approximately 1 hour following the manufacturer's recommendations. The membrane was hybridized with a 500 bp digoxigenin-labeled Aspergillus niger spo14 probe, which was synthesized by incorporation of digoxigenin-11-dUTP by PCR using primers spo14-5 (sense) and spo14-6 (antisense) shown below.

Forward primer
(spo14-5):
(SEQ ID NO: 8)
5′-CACTGGCAGCCAGGAGAAGC-3′
Reverse primer
(spo14-6):
(SEQ ID NO: 9)
5′-GGAGTGCTCGGTCGGAGTGC-3′

The amplification reaction (100 μl) was composed of 200 μM PCR DIG Labeling Mix (vial 2) (Roche Applied Science, Palo Alto, Calif., USA), 0.5 μM primers, EXPAND® High Fidelity Enzyme mix (vial 1) (Roche Applied Science, Palo Alto, USA), and 1 μl (100 μg/μl) of pHUda2368 as template in a final volume of 100 μl. The amplification reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 30 seconds and a 4° C. hold. PCR products were separated by 0.8% agarose gel electrophoresis using TAE buffer where a 0.5 kb fragment was excised from the gel and extracted using a QIAQUICK® Gel Extraction Kit. The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker. A strain, O74UVH, giving the correct integration at the spo14 loci (a hybridized band shifted from 3.3 kb to 4.5 kb) were selected for the subsequent experiments.

### Example 3: The Aspergillus niger Spo14 Gene Disruption in O73TYS

Protoplasts of Aspergillus niger strain O73TYS were prepared by cultivating the strain in 100 ml of YPG medium supplemented with 10 mM uridine at 32° C. for 16 hours with gentle agitation at 80 rpm. Pellets were collected and washed with 0.6 M KCl, and resuspended 20 ml 0.6 M KCl containing a commercial β-glucanase product (GLUCANEX™, Novozymes A/S, Bagsværd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 10 μg of pHUda2368 was added to 0.3 ml of the protoplast suspension, mixed gently, and incubated on ice for 30 minutes. Three ml of SPTC was added and the protoplast suspension was incubated for 20 minutes at 37° C. After the addition of 12 ml of 50° C. COVE-N top agarose, the mixture was poured onto the COVE-N plates and the plates were incubated at 30° C. for 7 days. The grown transformants were transferred with sterile toothpicks to Cove-N JP plates supplemented with 1.5 μM 5-Flouro-2-deoxyuridine (FdU), an agent which kills cells expressing the Herpes simplex virus (HSV) thymidine kinase gene (TK) harboring in pHUda2368. Single spore isolates were transferred to COVE-N-glyX plates.

Possible transformants of Aspergillus niger strain O73TYS containing the pHUda2368 to disrupt spo14 gene were screened by Southern analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) follows by manufacture's instruction.

Southern blot analysis was performed to confirm the disruption of the spo14 gene locus. Five μg of genomic DNA from each transformant were digested with SpeI and SphI. The genomic DNA digestion reactions were composed of 5 μg of genomic DNA, 1 μl of SpeI, 1 μl of SphI, 2 μl of 10×NEBuffer 4, and water to 20 μl. Genomic DNA digestions were incubated at 37° C. for approximately 16 hours. The digestions were submitted to 0.8% agarose gel electrophoresis using TAE buffer and blotted onto a hybond N+(GE Healthcare Life Sciences, Manchester, N.H., USA) using a TURBOBLOTTER® for approximately 1 hour following the manufacturer's recommendations. The membrane was hybridized with a 500 bp digoxigenin-labeled Aspergillus niger spo14 probe, which was synthesized by incorporation of digoxigenin-11-dUTP by PCR using primers spo14-5 (sense) and spo14-6 (antisense) shown above.

The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker. Strains, O835NC, O835ND and O835NE, giving the correct integration at the spo14 loci (a hybridized band shifted from 3.5 kb to 4.5 kb) were selected for the subsequent experiments.

### Example 4: Expression Evaluation of Spo14 Inactivated Strains from O73TYS in Shake Flask Culture

Aspergillus niger O835NC, O835ND & O835NE and their parent strain O73TYS were cultivated on COVE-N-glyX plates at 30° C. for about a few weeks. A sterile transfer pipette was used to punch a piece of small plugs from each plate, which were each inoculated into 100 ml of MSS medium in 500 ml flasks. The flasks were incubated at 30° C. for 3 days at 200 rpm. And then, 10 ml of culture broth was transferred to 100 ml of MU1 medium in 500 ml flasks. The flasks were incubated at 32° C. for 6 days at 200 rpm. Each culture was centrifuged at 5,000 rpm for 10 minutes in a 10 ml test tubes and culture supernatant was recovered for determining glucoamylase (AGU) productivities. Their enzyme activities (AGU activities) were measured followed by the methods described above; results are shown in the table below. The enzyme (AGU) productivities were determined by extrapolation from the generated standard curve and compared to the Aspergillus niger strain O73TYS set at 100%.

The spo14 gene disrupted strain gave 18-25% higher AGU productivity than a reference strain Aspergillus niger strain O73TYS in shake flasks (Table 1).

TABLE 1
AGU productivity in shake flask culture.

| | Relative protein |
|---|---|
| Sample Name | productivity (%) |
| A. niger O73TYS | 100% |
| A. niger O835NC | 122% |
| A. niger O835ND | 125% |
| A. niger O835NE | 118% |

### Example 5: Expression Evaluation of Spo14 Inactivated Strains from O73TYS in Lab-Scale Tank Cultivation

Aspergillus niger O835NC, O835ND & O835NE and their parent strain O73TYS were cultivated in lab-scale tanks under the fermentation method described in the Materials and Methods. Each culture sample was collected for determining glucoamylase (AGU) productivities. Their enzyme activities (AGU activities) were measured followed by the methods described above; results are shown in the table below. The enzyme (AGU) productivities per dosed glucose were determined by extrapolation from the generated standard curve and compared to the Aspergillus niger strain O73TYS set at 100%.

The spo14 gene disrupted strain gave 5-7% higher AGU productivity per dosed glucose than a reference strain Aspergillus niger strain O73TYS in lab-scale tanks (Table 2).

TABLE 2
AGU productivity in shake flask cultures.

| | Relative protein |
|---|---|
| Sample Name | productivity (/o) |
| A. niger O73TYS | 100% |
| A. niger O835NC | 105% |
| A. niger O835ND | 107% |
| A. niger O835NE | 106% |

### Example 6: Construction of Purpureocillium lilacinum Dextranase Gene (Pldex) Expression Vector pHUda2370

Plasmid pHUda2370 was constructed to contain Purpureocillium lilacinum dextranase gene (pldex) driven by Aspergillus niger neutral amylase promoter II (Pna2) and glucoamylase terminator (Tamg), the A. nidulans acetamidase gene (amdS) as a selectable marker, and the yeast Saccharomyces cerevisiae FLP recombinase gene (flp) driven by Aspergillus niger acid stable amylase promoter (PasaA) and the Aspergillus oryzae nitrate reductase terminator (Tniad).

A PCR product containing the pldex gene was generated using the following primers:

Primerpldex-1
(sense):
(SEQ ID NO: 10)
5′-ggatttagtcttgatcggatccaccatgcgttggcctggt-3′
Primerpldex-2
(antisense):
(SEQ ID NO: 11)
5′-gaaatggattgattgtcacgtgTTAttcaatgctccagtc-3′

The desired fragment was amplified by PCR in a reaction composed of approximately 100 ng of the plasmid DNA harboring pldex gene, 1 μl of Expand High Fidelity polymerase (Roche), 100 μM of primerpldex −1, 100 μM of primerpldex −2, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 2 minute; 1 cycle at 72° C. for 7 minutes; and a 4° C. hold. The resulting 1,824 bp PCR fragment was purified by 0.8% agarose gel electrophoresis using TAE buffer, excised from the gel, and extracted using a QIAQUICK® Gel Extraction Kit.

Plasmid pRika147 (described in example 9 in WO2012160093) was digested with BamHI and PmII, and purified by 0.8% agarose gel electrophoresis using TAE buffer, where a 10,512 bp fragment was excised from the gel and extracted using a QIAQUICK® Gel Extraction Kit. The purified 1,824 bp PCR fragment was fused to the 10,512 bp fragment in a reaction composed of 1 μl of the 10,512 bp fragment, 3 μl of the 1,824 bp fragment and 1 μl of 5× In-Fusion HD Enzyme Premix (In-Fusion® HD Cloning Kit/Clonetech). The ligation reaction was incubated at 50 deg C. for 10 minutes. Three μl of the mixture were transformed into DH5a chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda2370.

### Example 7: Introduction of Purpureocillium lilacinum Dextranase Gene (Pldex) Expression Vector pHUda2370 in Aspergillus niger Strains O73P66 and O74UVH

The pldex expression plasmids should be introduced at four pre-specified loci which are mannosyltransferase (alg2), glucokinase (gukA), acid stable amylase (asaA) and multicopper oxidase (mcoH) by flp recombinase.

Protoplasts of Aspergillus niger strain O73P66 and O74UVH were prepared by cultivating the strain in 100 ml of YPG medium at 32° C. for 16 hours with gentle agitation at 80 rpm. Pellets were collected and washed with 0.6 M KCl, and resuspended 20 ml 0.6 M KCl containing a commercial β-glucanase product (GLUCANEX™, Novozymes A/S, Bagsværd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 10 μg of pHUda2370 was added to 0.3 ml of the protoplast suspension, mixed gently, and incubated on ice for 30 minutes. Three ml of SPTC was added and the protoplast suspension was incubated for 20 minutes at 37° C. After the addition of 12 ml of 50° C. COVE top agarose supplemented with 50 μg/ml of 5′ fluorocytosine (5FC), an agent which kills cells expressing the Aspergillus niger cytosine deaminase (fcy1) gene harboring O73P66 and O74UVH, the mixture was poured onto the COVE plates and the plates were incubated at 30° C. for 10 days. The grown transformants were transferred with sterile toothpicks to Cove-2 plates supplemented with 10 μg/ml of 5′ fluorocytosine (5FC). Single spore isolates were transferred to COVE-N-glyX plates.

Possible transformants of Aspergillus niger strain either O73P66 and O74UVH containing the pHUda2370 to introduce pldex gene were screened by Southern analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) follows by manufacture's instruction.

Southern blot analysis was performed to confirm the introduction of the pldex gene at four pre-specified locus (alg2, gukA, asaA, mcoH). Five μg of genomic DNA from each transformant were digested with SacII. The genomic DNA digestion reactions were composed of 5 μg of genomic DNA, 0.5 μl of SacIII, 2 μl of 10×NEBuffer 4, and water to 20 μl. Genomic DNA digestions were incubated at 37° C. for approximately 16 hours. The digestions were submitted to 0.8% agarose gel electrophoresis using TAE buffer and blotted onto a hybond N+ (GE Healthcare Life Sciences, Manchester, N.H., USA) using a TURBOBLOTTER® for approximately 1 hour following the manufacturer's recommendations. The membrane was hybridized with a 500 bp digoxigenin-labeled pldex probe, which was synthesized by incorporation of digoxigenin-11-dUTP by PCR using primers pldex-3 (sense) and pldex-4 (antisense) shown below.

Forward primer
(pldexC-3):
(SEQ ID NO: 12)
5′-atgcgttggcctggtaattt-3′
Reverse primer
(pldexC-4):
(SEQ ID NO: 13)
5′-gatggtctcgtagacaaacg-3′

The amplification reaction (100 μl) was composed of 200 μM PCR DIG Labeling Mix (vial 2) (Roche Applied Science, Palo Alto, Calif., USA), 0.5 μM primers, EXPAND® High Fidelity Enzyme mix (vial 1) (Roche Applied Science, Palo Alto, USA), and 1 μl (100 μg/μl) of pHUda2370 as template in a final volume of 100 μl. The amplification reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 30 seconds and a 4° C. hold. PCR products were separated by 0.8% agarose gel electrophoresis using TAE buffer where a 0.3 kb fragment was excised from the gel and extracted using a QIAQUICK® Gel Extraction Kit. The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker. Strains C5559-2370-1, 8, 9 and C5559-2368-2370-1, 3 6, generated from O73P66 and O74UVH, respectively, giving the correct integration were selected for the subsequent experiments.

### Example 8: PLDEX Expression Evaluation in Shake Flask Culture

Aspergillus niger strains C5559-2370-1, 8, 9, C5559-2368-2370-1, 3 6 & O73P66 and O74UVH were cultivated on COVE-N-glyX plates at 30° C. for about a few weeks. A sterile transfer pipette was used to punch a piece of small plugs from each plate, which were each inoculated into 100 ml of MSS medium in 500 ml flasks. The flasks were incubated at 30° C. for 3 days at 200 rpm. And then, 10 ml of culture broth was transferred to 100 ml of MU1 glu medium in 500 ml flasks. The flasks were incubated at 30° C. for 5 days at 200 rpm. Each culture was centrifuged at 5,000 rpm for 10 minutes in a 10 ml test tubes and culture supernatant was recovered for determining dextranase productivities. The dextranase productivity assay was performed using a Quick Start™ Bradford Protein Assay Kit (Bio-Rad inc.). Culture supernatants were diluted appropriately in distilled water. A bovine serum albumin (WAKO cat number 519-83921) was diluted using several steps starting with a 0.5 mg/ml concentration and ending with a 0.1 mg/ml concentration in the distilled water. Five μl of each dilution including standard were transferred to a 96-well flat bottom plate. 250 μl of 1× Dye Reagent solution were added to each well and then incubated at room temperature for 5 minutes. The endpoint of the reaction was measured at 595 nm. Whole protein productivities were determined by extrapolation from the generated standard curve.

The average dextranase productivities of spo14 gene disrupted strains from a strain O74UVH gave 60-65% higher than those of the strains from O73P66. (Table 3)

TABLE 3
Dextranase productivity in shake flask culture (triplicate). The
productivity of C5559-2370-1 in shake flask is set as 100%.

| | Relative protein |
|---|---|
| Sample Name | productivity (%) |
| A. niger C5559-2370-1 | 100% |
| A. niger C5559-2370-8 | 95% |
| A. niger C5559-2370-9 | 108% |
| A. niger C5559-2368-2370-1 | 165% |
| A. niger C5559-2368-2370-3 | 170% |
| A. niger C5559-2368-2370-6 | 160% |

1-21. (canceled)

22: A filamentous fungal host cell comprising a heterologous polynucleotide encoding a secreted polypeptide of interest and comprising an inactivated spo14 gene or homologue thereof, wherein said spo14 gene or homologue thereof encodes a phospholipase D having an amino acid sequence at least 80% identical to SEQ ID NO: 3.

23: The host cell of claim 22 which is of a genus selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma.

24: The host cell of claim 22 which is an Aspergillus cell.

25: The host cell of claim 22 which is an Aspergillus awamori, Aspergillus foetidus, Aspergillus fumigatus, Aspergillus japonicus, Aspergillus nidulans, Aspergillus niger or an Aspergillus oryzae cell.

26: The host cell of claim 22, wherein the secreted polypeptide of interest is an enzyme.

27: The host cell of claim 22, wherein the secreted polypeptide of interest is a hydrolase, isomerase, ligase, lyase, oxidoreductase, or transferase.

28: The host cell of claim 22, wherein the secreted polypeptide of interest is an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

29: The host cell of claim 22, wherein the phospholipase D has an amino acid sequence at least 90% identical to SEQ ID NO: 3.

30: The host cell of claim 22, wherein the phospholipase D has an amino acid sequence that comprises or consists of SEQ ID NO: 3.

31: The host cell of claim 22, wherein the spo14 gene or homologue thereof has a genomic nucleotide sequence at least 90% identical to SEQ ID NO: 1; preferably at least 75%, 80%, 85%, 90%, 95%, 96%, 97%, 98% or most preferably at least 99% identical to SEQ ID NO:1.

32: The host cell of claim 22, wherein the spo14 gene or homologue thereof has a genomic nucleotide sequence that comprises or consists of SEQ ID NO: 1.

33: The host cell of claim 22, wherein the spo14 gene or homologue thereof comprises or consists of a genomic nucleotide sequence, the cDNA sequence of which is at least 90% identical to SEQ ID NO: 2.

34: The host cell of claim 22, wherein the spo14 gene or homologue thereof comprises or consists of a genomic nucleotide sequence, the cDNA sequence of which comprises or consists of SEQ ID NO: 2.

35: A method of producing a secreted polypeptide of interest, said method comprising cultivating the filamentous fungal host cell of claim 22.

36: The method of claim 35, further comprising recovering the secreted polypeptide of interest.

37: A method of producing a filamentous fungal host cell having an improved yield of a secreted heterologous polypeptide of interest, said method comprising the following steps in no particular order:
a) transforming a filamentous fungal host cell with a heterologous polynucleotide encoding the secreted polypeptide of interest; and
b) inactivating an spo14 gene or a homologue thereof in the filamentous fungal host cell, wherein said spo14 gene or a homologue thereof encodes an phospholipase D having an amino acid sequence at least 80% identical to SEQ ID NO: 3.
